(19) United States
(12) Patent Application Publication
(10) Pub. No.: US 2021/0330843 A1
(43) Pub. Date: Oct. 28, 2021

(54) DISINFECTION METHOD AND DISINFECTION DEVICE

(71) Applicant: CLEAMIX OY, Riistavesi (FI)

(72) Inventor: Harri HAKKARAINEN, Tallinn (EE)

(21) Appl. No.: 17/274,340

(22) PCT Filed: Sep. 5, 2019
(86) PCT No.: PCT/FI2019/050633

§ 371 Date: Mar. 8, 2021

(30) Foreign Application Priority Data
Sep. 9, 2018 (FI) 20187126

(51) Int. Cl. A61L 2/20; A61L 2/26

(52) U.S. Cl. CPC A61L 2/208; A61L 2/26; A61L 2202/26; A61L 2202/14; A61L 2202/15; A61L 2202/121; A61L 2202/11; A61L 2202/25

(57) ABSTRACT

The present invention relates to a disinfection method to be used for cleaning. The invention is characterized in that hydrogen peroxide (H₂O₂) is drained/transferred from one or more tanks (16) by a liquid pressure created by one or more pumps or gravity, through one or more draining members (6) to one or more draining devices (5), which draining device (5) drains hydrogen peroxide to one or more onto one or more evaporation top surfaces (4ea) of an evaporation member (4e) of a warming/heating device (4), the evaporation top surface (4ea) is at an evaporation angle (4d) of 1 to 30 degrees in relation to the horizontal plane so that the end on the draining device (5) side draining end (4c) is higher than an opposite end of the draining device (5) of the warming/heating device (4), whereby hydrogen peroxide spreads by gravity on the evaporation member (4e) where the hydrogen peroxide (H₂O₂) turns into hydrogen peroxide gas, by means of which hydrogen peroxide gas the disinfection is performed, the evaporation member (4e) consists of one or more fibreglass braidings, the airflow area is reduced by one or more air guides (2) on the top side of the evaporation top surface (4ea) of the evaporation member (4ea) of the warming/heating device (2) towards the gas discharge end (4d) whereby the airflow rate is increased towards the gas discharge end (4d).

### OBJECT OF THE INVENTION

The present invention relates to a disinfection method for use for cleaning in targets to be cleaned, such as the living environment of people and animals, as well as in the growing/storage environment of human and animal food, or in the transport/moving environment of people and animals. The invention additionally relates to a disinfecting device for use for cleaning in targets to be cleaned, such as the living environment of people and animals, as well as in the growing/storage environment of human and animal food, or in the transport/moving environment of people and animals.

TARGETS TO BE CLEANED IN MORE DETAIL

Used for exterminating microbes and bacteria. Any premises or things are cleaned, which have been contaminated by microbes, bacteria, or germ growth, such as hospitals

Disinfecting is needed:
At hospitals for cleaning the premises. For controlling hospital bacteria and contagious diseases.
For cleaning ambulances and patient transportation equipment.
For cleaning the transport equipment and other gear of fire and rescue services.
For cleaning air conditioning systems of buildings.
For cleaning laboratory spaces (e.g. safety cabinets).
In food industry, for cleaning rooms, machines, and transport equipment.
Manufacture of biofuels—Reducing product loss. Contaminated target, only, is cleaned.
In defence forces, bioterrorism, biowarfare, the contaminated targets/equipment are cleaned.
In security applications, safety applications, the transport equipment and other gear, such as clothing, are cleaned.
Animal diseases/animal production premises. Contaminated premises are cleaned.
Transport logistics (ships, aeroplanes etc.), contaminated transport equipment is cleaned.
For pest control and micro-organisms control, vermin control, such as ant control, silver fish control, etc.

Common nouns for nuisances to be disinfected and their targets.
Bacteria, microbes, viruses, and pests/vermin. For example at hospitals, households, shops, laboratories, premises/spaces used by people and animals in general.
Bacteria, microbes, viruses, and pests/vermin. Cleaning of premises/areas after contamination by, for example, biological warfare/terrorism. Following a natural disaster (destroying living material, for example, for disinfecting bodies, whereby bodies cannot spread bacteria, microbes, or viruses).
Bacteria, microbes, viruses, and pests/vermin. Different kinds of channels for moving material, route networks, tunnels for transporting material, such as ventilation channels, water pipelines (when empty), sewer pipes.
Bacteria, microbes, viruses, and pests/vermin. Vehicles on land, water, and air, such as ambulance vehicles, fire-fighting vehicles, buses, cars, aeroplanes, ships, rockets, work machines.
Bacteria, microbes, viruses, and pests/vermin.

### PRIOR ART

Currently, weak hydrogen peroxide is used for cleaning, by spraying the substance in question to the space directly onto the target(s) to be cleaned.

The problem is non-spreading, does not spread everywhere. Corrosion effect, because hydrogen peroxide is corrosive in its liquid form. Adjusting the amount of hydrogen peroxide content is difficult, because dosing hydrogen peroxide evenly on the surface to be cleaned is impossible to achieve. The hydrogen peroxide content is uneven, resulting in a varying cleaning time. Close to the moisture point, an imprecise dosage leads to the hydrogen peroxide condensing back to liquid, which leads to liquid hydrogen peroxide remaining on the cleaned target, corroding the cleaned target. Cleaning by hydrogen peroxide is presently slow, imprecise and consequently expensive work. In addition, part of the cleaned target will not be evenly cleaned, if at all.

From publication JP 2003339829 A (Figure, machine translation paragraphs [0013]-[0024]), a disinfection method and disinfection device for the purpose of cleaning are known. In the method, hydrogen peroxide is sprayed from a container at a liquid pressure created by a pump through a spraying member to an evaporation member where also a hot carrier gas stream is led. In the evaporation member, hydrogen peroxide turns into hydrogen peroxide gas. To blow the carrier gas stream, a blower is used.

In publication JP 2003339829 A, the evaporation member is formed of a long, straight and open space with closed walls, the evaporation member being referred to as a flushing area/zone in the publication, to which flushing area/zone air and hydrogen peroxide solution are mixedly sprayed, due to which hydrogen peroxide comes as small droplets. The flushing area/zone is heated from the outside by an electric heater. From the machine translation, “the flushing area/zone, where downstream of a spray nozzle the electric heater is installed on flushing area/zone with an enlarged diameter, the electric heater is sufficiently long, the hydrogen peroxide solution evaporates into fine particles. Furthermore, the flushing area/zone is cylindrical or trumpet shaped, when viewed from the side, and is preferably vertically or horizontally. The flushing area/zone has no evaporation surface.

From publication EP2650023A1 (the entire publication; in particular paragraphs [0014]-[0024]; figures), a disinfection method and disinfection device for cleaning are known. In the method, hydrogen peroxide is sprayed from a tank at a liquid pressure produced by a pump through a spraying member to a evaporation member where also a hot carrier gas stream is led. In the evaporation member, hydrogen peroxide turns into hydrogen peroxide gas. FIG. 4, in particular, shows that the evaporation member is a closed tank that has no evaporation surface in-side.

From publication US2004265459A1 (paragraphs [0006]-[0009], [0065]-[0068]; figure), a disinfection method and disinfection device for cleaning are known. In the method, a disinfectant (such as hydrogen peroxide) is sprayed from a tank at a liquid pressure produced by a pump through a spraying member to a evaporation member where also hot carrier gas stream is led. In the evaporating member, hydrogen peroxide is vaporized. In the publication is not mentioned that expressly a blower is used for blowing hot carrier gas. FIG. 1, in particular, shows that the evaporation member is a closed container that does not have any evaporation surface inside, just guide plates to mix the carrier gas and acetic acid.

In all the publications JP 2003339829 A, EP2650023A1 and US2004265459A1, the evaporation member is formed of a closed tank-like structure inside the tank-like structure of which there is no evaporation surface or means. In all the publications JP 2003339829 A, EP2650023A1 and US2004265459A1, hydrogen peroxide is sprayed by means of a liquid pressure created by a pump to an evaporation member where the hydrogen peroxide is vaporised.

### OBJECT OF THE INVENTION

The object is that disinfection is carried out by hydrogen peroxide gas (H₂O₂), whereby no corrosion takes place, because hydrogen peroxide (H₂O₂) is not corrosive in the gaseous state.

The above disadvantages may be eliminated and the above goals be reached by the inventive disinfection method which is characterised by what is disclosed in the characterising part of claim 1, and the preferred embodiments of the methods are disclosed in claims 2 to 9. The disinfection device according to the invention is characterised by what is disclosed in the characterizing part of claim 10, and the preferred embodiments of a fastening element are disclosed in dependent claims 11 to 14.

As the most important benefits of the invention, it may be mentioned that the invented disinfection method provides a precise dosage of hydrogen peroxide gas on a target to be cleaned. The exact amount of hydrogen peroxide may be precisely dosed by draining hydrogen peroxide from one or more nozzles as a liquid stream on one or more warming/heating devices having one or more evaporation members which is the evaporation member. The evaporation member is the upper part of the warming/heating device, on the top surface of which to a draining end hydrogen peroxide is drained from one or more draining devices. The evaporation member consist of a fibreglass braiding. The evaporation top surface of the evaporation member is at an evaporation angle of 1 to 30 degrees, as seen from the side, so that an evaporation draining end higher than an evaporation gas end, whereby hydrogen peroxide drains downhill and is evenly spread on the entire evaporation top surface of the evaporation member following the draining device on which evaporation top surface hydrogen peroxide is gasified into hydrogen peroxide gas by means of which hydrogen peroxide gas cleaning is best performed controllably by one or more blow channels/blow pipes. The shape of the cross-section of the blow channel may differ from round, the cross-section may have a known shape such as a square, rectangle, oval, triangle, polygon etc. On the evaporation top surface, hydrogen peroxide evaporates fast into hydrogen peroxide gas as the warming member under-side of the evaporation surface warms up the evaporation surface. The airflow on the top side the evaporation surface is faster than the airflow under-side, whereby hydrogen peroxide gasifies fast, because the faster airflow on the top side tends to suck the vaporizing hydrogen peroxide with it. The airflow on the top side of the evaporation surface is colder than the airflow under side, due to which airflow swirls, so-called turbulence, is created on the top side of the evaporation member, which speeds up the gasifying of hydrogen peroxide. It is obvious that the use of the invented disinfection method results in great cost savings in cleaning.

GENERAL INFORMATION ON HYDROGEN PEROXIDE (Wikipedia) https://fi.wikipedia.org/wiki/Vetyperoksidi #Aiheesta_muualla

Hydrogen oxide (sometimes hydrogen superoxide) H₂O₂ is one of the oxides of hydrogen, whose CAS number is 7722-84-1. The other, more common oxide of hydrogen, is water (H₂O).

Properties

Hydrogen peroxide is a strongly oxidising agent. When warmed, it decomposes into water and oxygen whereby energy is released in the process at the same time. Some metals and impurities act as catalysts of the decomposing process. Hydrogen peroxide liquid may be stabilized with, for example phosphorus, sulphur, boron, or citric acid, acetanilide, or acetophenetide when the aim is to slow down the decomposing process.

Hydrogen peroxide of less than 85 percent does not burn, but it is a strongly oxidising agent, so hydrogen peroxide together with a burning agent causes a serious danger of burning or explosion. Hydrogen peroxide over 85% burns, as it is decomposing, with a blue flame, so its decomposing reaction differs from that of hydrogen peroxide solutions weaker than that.

When decomposing, hydrogen peroxide turns into water and oxygen.

2 H₂O₂→2 H₂O +O₂.

Hydrogen peroxide is unstable and decomposes by itself, but the reaction is extremely slow. The reaction may be accelerated by the use of a catalyst (such as manganese dioxide). In hydrogen peroxide, the oxidation number of oxygen is −I, in decomposition products O (O₂) and −II (H₂O). So, a disproportionation takes place in the reaction, with the oxidation number both increasing and decreasing.

The capability of bleaching and disinfecting of hydrogen peroxide is based on a very reactive free oxygen atom formed in the decomposition process.

Uses

Hydrogen oxide has been used as the oxidant of the fuel used for carrier rockets.

Industrial uses of hydrogen peroxide include bleaching of pulp and textiles and as a disinfectant in medicine and food industries. The hydrogen peroxide used in the industry usually has 35 or 50 percent of hydrogen peroxide, but other concentrations are available as well. For disinfection, hydrogen peroxide of 100 percent may also be used.

Hydrogen peroxide of a low (less than 5 percent) concentration is used in cosmetics, such as hair bleaching, and for example as a cleaning solution of contact lenses, and in disinfecting wounds. The capability of disinfection is based on decomposition of hydrogen peroxide blood acting as the catalyst. The oxygen being released kills bacteria.^{[2][3]}

Hydrogen peroxide is used rather extensively these days, because its potential release into the air or nature elsewhere only causes short-term harm. Having been released, it decomposes relatively quickly into water and oxygen and does not therefore cause long-term problems for the environment or population.

### LIST OF FIGURES

In the following the invention is explained in detail with reference to the accompanying figures, in which

FIG. 1 is a sectional perpendicular side view of a disinfection device body according to the invented disinfection method,

FIG. 2 is a perpendicular view from the top of the disinfection device body of FIG. 1,

FIG. 3 is a perpendicular side view of warming/heating device inside the disinfection device body of FIGS. 1 and 2,

FIG. 4 is a is a perpendicular top view of the warming/heating device of FIG. 3,

FIG. 5 is a perpendicular end view of the warming/heating device of FIGS. 3 and 4,

FIG. 6 is an enlarged perpendicular end view of the warming/heating device of FIG. 5,

FIG. 7 is a perpendicular end view of an assembled evaporation member of the warming/heating device of FIG. 6,

FIG. 8 is an exploded perpendicular end view of the evaporation member of FIG. 7,

FIG. 9 is perpendicular side view of the evaporation member of FIGS. 7 and 8 at a skew angle, where the evaporation member is installed to the disinfection device body, to the left end of FIG. 9 at the top side, there will be a draining device,

FIG. 10 is a perpendicular end view of a warming member of the warming/heating device of FIGS. 5 and 6,

FIG. 11 is a perpendicular end view of a warmer of the warming/heating device of FIGS. 5 and 6,

FIG. 12 is a perpendicular end view of a cooler of the warming/heating device of FIGS. 5 and 6,

FIG. 13 is a perpendicular side view of a draining device of hydrogen peroxide, inside the disinfection device body of FIGS. 1 and 2, the hydrogen peroxide liquid drains from underside the draining device through draining channels opening on the right side of the Figure,

FIG. 14 is a perpendicular detailed view form the draining direction of the draining device of FIG. 13,

FIG. 15 is a perpendicular view from the draining direction of the draining device of FIGS. 13 and 14 as seen perpendicularly from the draining direction,

FIG. 16 is a perpendicular view from underside of the draining device of FIGS. 13, 14 and 15,

FIG. 17 is a perpendicular view from the top of the disinfection device body of FIGS. 1 and 2, placed in a usage space,

FIG. 18 is a perpendicular front view of the disinfection device body of FIG. 17, placed in a usage space, the usage space closed, in the usage space, on the left, a suction channel/suction pipe is drawn as bent into a transport position, and on the right, a blow channel/blow pipe bent into a transport position,

FIG. 19 is a perpendicular front view of the disinfection device body of FIG. 18, placed in a usage space, the usage space opened,

FIG. 20 is a perpendicular view from the top of the disinfection device body of FIGS. 18 and 19, placed in a usage space,

FIG. 21 is a perpendicular front view of the disinfection device body of FIGS. 18, 19, and 20 placed in a usage space, the usage space opened, in the usage space, on the left, a suction channel/suction pipe is drawn opened into use position/cleaning position, and on the right, a blow channel/blow pipe opened into a use position/cleaning position,

FIG. 22 is a perpendicular front view of the disinfection device body of FIG. 21 placed in a usage space, the usage space opened, in the usage space, on the left, is drawn a hydrogen peroxide tank suspended from the cover of the usage space, from where hydrogen peroxide drains under gravity to the draining device,

FIG. 23 is a view of a second disinfection device according to the invention, a disinfection device body placed in a usage space, as seen perpendicularly from the top, the usage space opened, in the usage space, on the left, a suction channel network/suction pipes are drawn opened into use position/cleaning position, and on the right, a blow channel system/blow pipes opened into a use position/cleaning position,

FIG. 24 is a view of the disinfection device body of FIG. 23 placed in a usage space, as seen perpendicularly from the top, the usage space opened, in the usage space, on the left, a suction channel network/suction pipes are drawn opened into use position/cleaning position, and on the right, a blow channel network/blow pipes opened into a use/cleaning position, the suction channels/suction pipes and blow channels/blow pipes pass through a gas tight partition wall to the space to be cleaned,

FIG. 25 is a view of a third disinfection device of the invention, the disinfection device body placed in a usage space, as seen perpendicularly from the front, the usage space opened, in the usage space, on the left, a suction channel/suction pipe is drawn opened into use position/cleaning position, and on the right, a blow channel/blow pipe opened into a use/cleaning position, the usage space has a pressure blower to blow hydrogen peroxide gas to the blow channels/blow pipes,

FIG. 26 is a view of a fourth disinfection device of the invention, disinfection device bodies placed in a disinfection cabinet, as seen perpendicularly from the front, in FIG. 26 one place is missing a disinfection device body, the Figure illustrates that desired number of disinfection device bodies may be placed in a disinfection cabinet, whereby the disinfection power of the disinfection cabinet is adjustable,

FIG. 27 is a perpendicular view from the top of a fifth disinfection device of the invention, disinfection device bodies placed in a star shape.

FIG. 28 is a perpendicular side view of a sixth disinfection device of the invention, the disinfection device body in cross section, there are two successive blowers inside the disinfection device body.

FIG. 29 is a perpendicular side view of a seventh disinfection device of the invention, disinfection device body in cross section, there is a blower inside the disinfection device body, placed at the air discharge end whereby the blower generates a vacuum to the disinfection device body,

FIG. 30 is a perpendicular side view of an eighth disinfection device of the invention, disinfection device body in cross section, there is a blower inside the disinfection device body, placed at the air discharge end whereby the blower generates a vacuum to the disinfection device body, there is an adjustable inward relief valve at the suction end of the body.

### DETAILED DESCRIPTION OF THE INVENTION

The invention shown in the accompanying figures and the associated parts are not shown in scale but the figures are schematic, illustrating the structure and operation of the preferred embodiment of the invention and its parts in principle.

The parts and points of the disinfection device, shown in the figures.

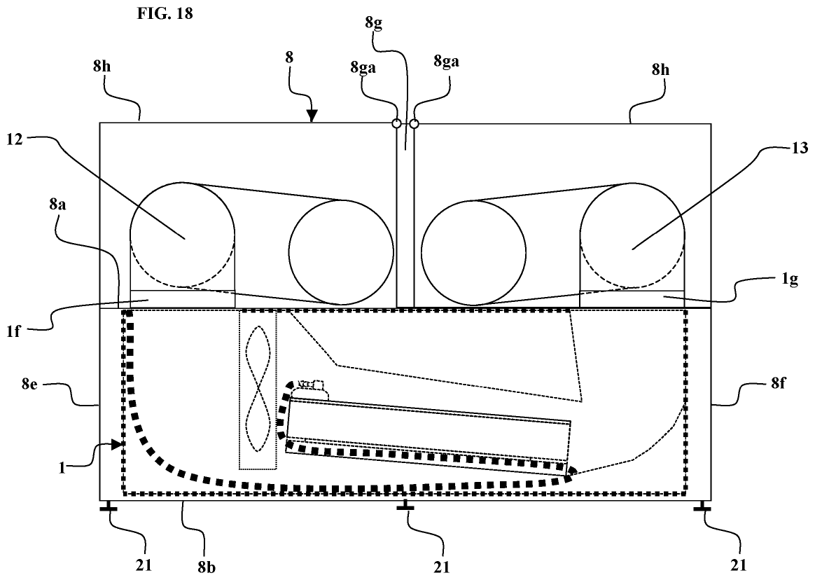

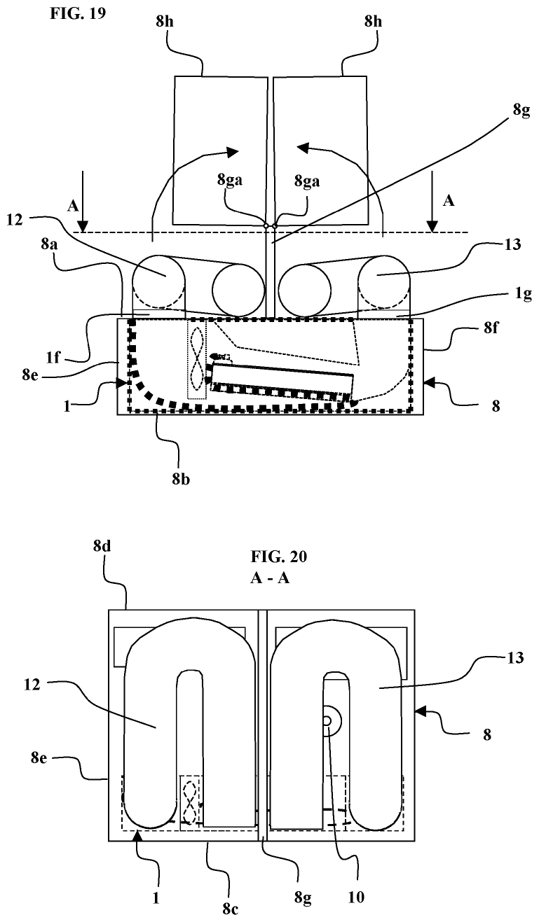

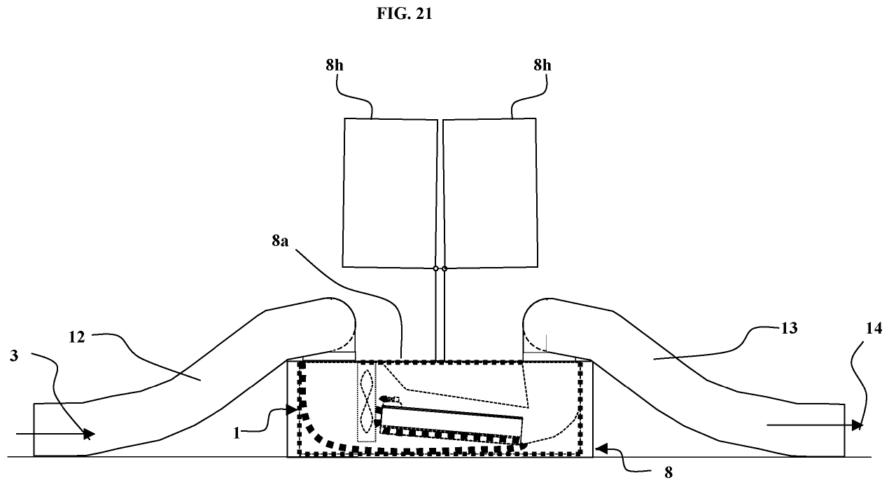

In the figures, the disinfection device body 1 is a closed, rectangular box made of metal, preferably stainless steel, on the top side la of which at the suction end 1d there is one or more suction opening/suction passages if, from which suction opening/suction passage if air/gas may enter inside the disinfection device body 1, said air/gas being used inside the disinfection device body 1 to vaporize hydrogen peroxide (H₂O₂). On the top side la of the disinfection device 1 at the blowing end 1e there is one or more blowing opening/blowing passages 1g from which blowing opening/blowing passage 1g hydrogen peroxide (H₂O₂) hydrogen peroxide gas 14 may access the target(s) to be cleaned directly or through a desired pipe or another passage/channel to one or more desired target(s). Unlike in the figures, the suction opening/suction passage if and blowing opening/blowing passage 1g may be located at an end/ends, underside, or side of the disinfection device body 1, the location may be freely chosen as needed because the blower 2 carries out the circulation of air or gas and air mixture. One or more suction channel/suction pipes 12 and blowing channel/blowing pipe 13 may be fastened as known the best rotatably, as shown in FIGS. 18, 19, 20, 21, 22, 23, 24 and 25, to the suction opening/suction passage if and blowing opening/blowing passage 1g.

The disinfection device body 1 has a top side 1a, underside 1b, side 1c, suction end 1d, blowing end 1e, suction opening/suction passage if, blowing opening/blowing passage 1g.

A blowing guide 1ga is formed in the figures of bent sheet metal, preferably stainless, fixed inside the disinfection device body 1 at the blowing end 1e.

One or more blowers 2 to blow clean air or to circulate air to be cleaned. The blower/blowers 2 suck suction air 3 inside the disinfection device body 1 through one or more suction openings/suction passages if at the suction end 1d.

The blower 2 is the best known axial blower, centrifugal blower or a similar device moving gas/air, which sucks gas/air in the direction shown by the arrow 3. Most preferably filtered air, the filter/cleaner known from the construction industry is not shown in the figures. Suction air 3, which suction air 3 may be air, nitrogen, argon, or a mixture of the aforementioned. The blower/blowers 2 blow suction air 3 to the draining end 4c of the hydrogen peroxide of the warming/heating device 4, being almost at horizontal plane as seen from the side.

To blow suction air 3, an air guide/pipe system may also be used to bring in air for the warming/heating device 4. With the same technology, a single large blower may be used, by means of which suction air 3 is blown to a plurality of warming/heating devices 4. for example in the structure solution of FIG. 27 one large blower is used to blow suction air 3 to eight disinfection device bodies 1 and the warming/heating devices 4 therein.

The warming/heating device 4 has a top side 4a, underside 4b, draining end 4c, gas discharge end 4d, and side 4e.

The evaporation member 4e is the topmost part of the warming/heating device 4, on the top surface of which to the draining end 4c hydrogen peroxide is drained from one or more draining devices 5.

The evaporation member 4e is formed of a fibreglass braiding. The evaporation top surface 4ea of the evaporation member 4 is at an evaporation angle 4ej of 1 to 30 degrees as seen from the side so that the evaporation draining end 4ec is higher than the evaporation gas end 4ed whereby hydrogen peroxide drains downhill and is evenly spread on the entire evaporation top surface 4ea following the draining device 5 of the evaporation member 4e.

The evaporation member 4e has an evaporation top surface 4ea, evaporation bottom surface 4eb, evaporation draining end 4ec, evaporation gas end 4ed, evaporation member side 4ee, and fibreglass braiding 4ef.

The fibreglass braiding 4ef is of known crosswise woven fibreglass braiding 4ef, which may also be referred to as a fibreglass mat. The density of the fibreglass braiding 4ef is such that air may pass through the fibreglass braiding, the thickness of the fibreglass braiding 4ef is 0.5 to 3 mm depending on the area of the evaporation member 4e.

In FIG. 4, a top frame 4eg has in the figures a rectangular opening 4ek to the evaporation top surface 4ea, from the area of this opening 4ek hydrogen peroxide may vaporize. The shape of the opening 4ek may differ from the rectangle of the figures, the shape as seen from above may be a cone or oval, or of another known shape.

In the Figures, a bottom frame 4eh has a rectangular opening 4ek, from the area of this opening 4ek the air from the blower 2 get to vaporize hydrogen peroxide from underside.

A net 4ei is a net at best made of stainless steel with a mesh size of 2 to 5 mm and wire thickness 0.3 to 1.0 mm, the net 4ei shape as seen from above is preferably square. Unlike in the Figures, the fibreglass braiding 4ef may be glued by heat-resistant glue to the top frame 4eg whereby the bottom frame 4eh will not be needed. On top of the fibreglass braiding 4ef there is a net 4ei which prevents an uncontrollable draining of hydrogen peroxide.

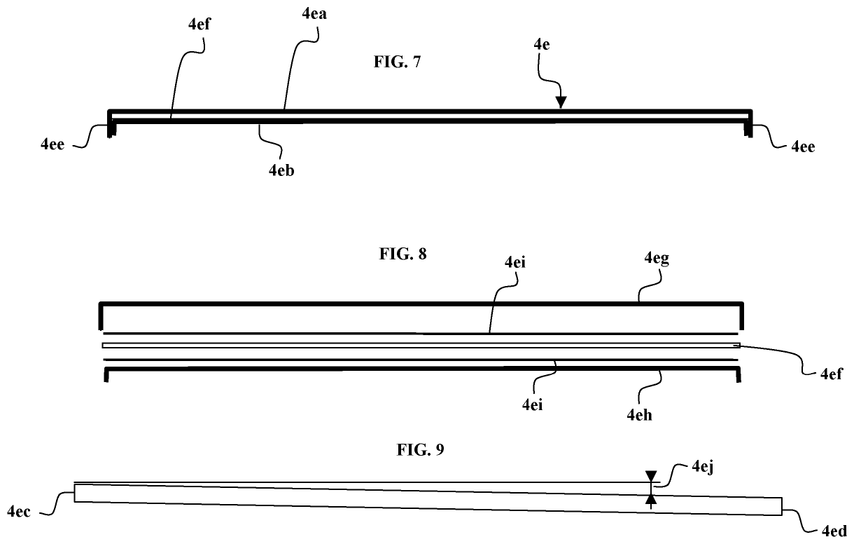

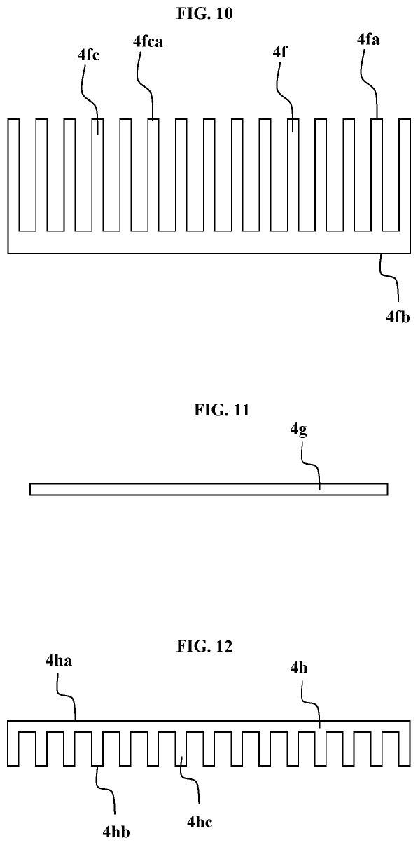

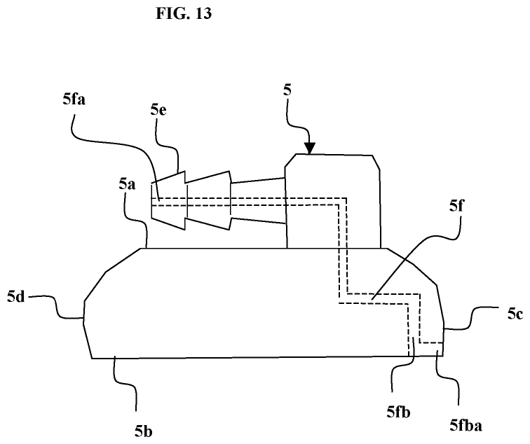

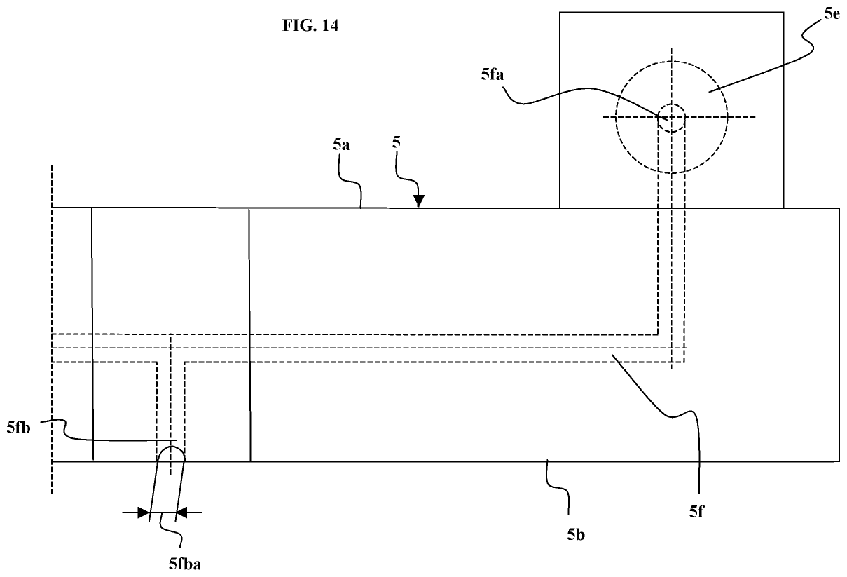

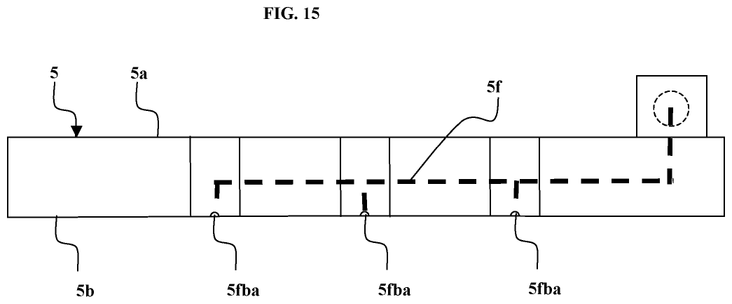

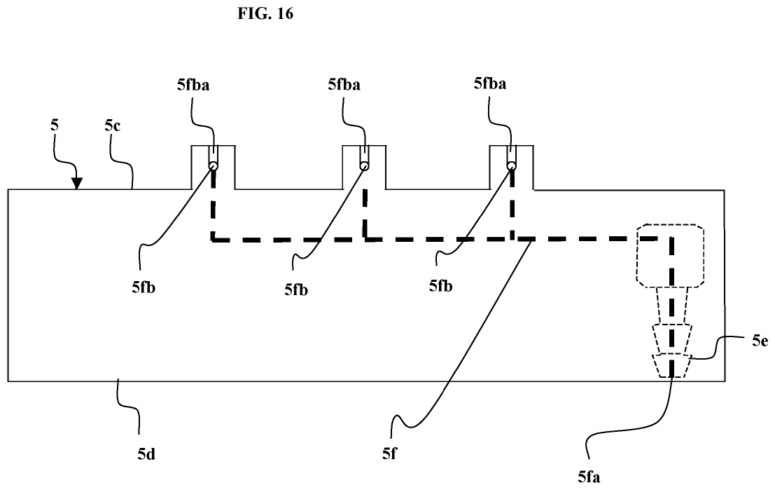

FIG. 8 shows that the net 4ei is at the top side and underside of the fibreglass braiding 4ef whereby the fibreglass braiding 4ef is pressed between the nets 4ei, pressed by the top frame 4eg and bottom frame 4eh, whereby fastening by glue is not needed. The top frame 4eg and bottom frame 4eh may be fixed to one another by known rivets, for example, preferably the downward bent sides (in FIGS. 7 and 8) of the bottom frame 4eg and bottom frame 4eh are doubled to an internal angle of 80 to 89 degrees whereby the top frame 4eg and bottom frame 4eh adhere to each other on their sides that are bent over by a compression joint connection.

In the figures, the warming member 4f is an aluminium piece, equipped with warming top side 4fa warming ribs 4fc.

The warming member 4f has a warming top side 4fa, warming underside 4fb, warming rib 4fc and in it a warming rid end 4fca.

In the figures, the warmer 4g is a warming plate continuously adjustable by electricity (electric energy), made of known electric elements by a known method, such as electric elements of the kind used in electric stoves, whose temperature may be continuously adjusted, as known.

A cooler 4h, the lowest part of the warming/heating device 4, the task of the cooler 4h is to manage the temperature of the warmer 4g by cooling the warmer 4g from the underside. The cooler 4h has a cooling top side 4ha and a cooling underside 4hb. In the figures, the cooler 4h is an aluminium piece, equipped with cooling underside 4hb cooling ribs 4hc. The cooler 4h has a cooling top side 4ha, a cooling underside 4hb, and one or more cooling ribs 4hc.

The draining device 5 of hydrogen peroxide is in the figures a piece manufactured by printing it from plastic, which has a draining pipe connector 5e and draining channel 5f for a draining pipe 6.

The draining device 5 has a draining top side 5a, a draining underside 5b, a draining side 5c, a blower side 5d, a draining pipe connector 5e.

The draining lower sider 5b hydrogen peroxide drains to the evaporation member 4e from the draining lower side 5b, more specifically to the draining side 5c. The draining side 5c is the side of the evaporation gas end 4ed, that is, the lower side of the evaporation member 4e. The blower side 5d is the side of the blower 2 side.

The draining channel 5f is one or more openings inside the draining device 5, which branches out to a plurality of openings. The draining channel 5f starts at one or more draining pipe connectors Se which is an inlet end 5fa of hydrogen peroxide and the draining channel 5f ends at an outlet draining end 5fb of hydrogen peroxide, in which outlet draining end of hydrogen peroxide 5fb has one of more draining guides 5fba of hydrogen peroxide.

The draining channel 5f has an inlet end 5fa of hydrogen peroxide and outlet draining end 5fb of hydrogen peroxide.

At the hydrogen peroxide outlet draining end 5fb there is a hydrogen peroxide draining guide 5fba. In the Figures, the hydrogen peroxide draining guide 5fba is a groove parallel to the draining bottom side 5b, along which hydrogen peroxide drains and spreads on the evaporation top side 4ea of the evaporation member 4e. In the Figures, there are three hydrogen peroxide draining guides 5fba but unlike in the Figures , there may be one or more pieces of them depending on the width of the warming/heating device 4, that is, the width of the evaporation surface of hydrogen peroxide.

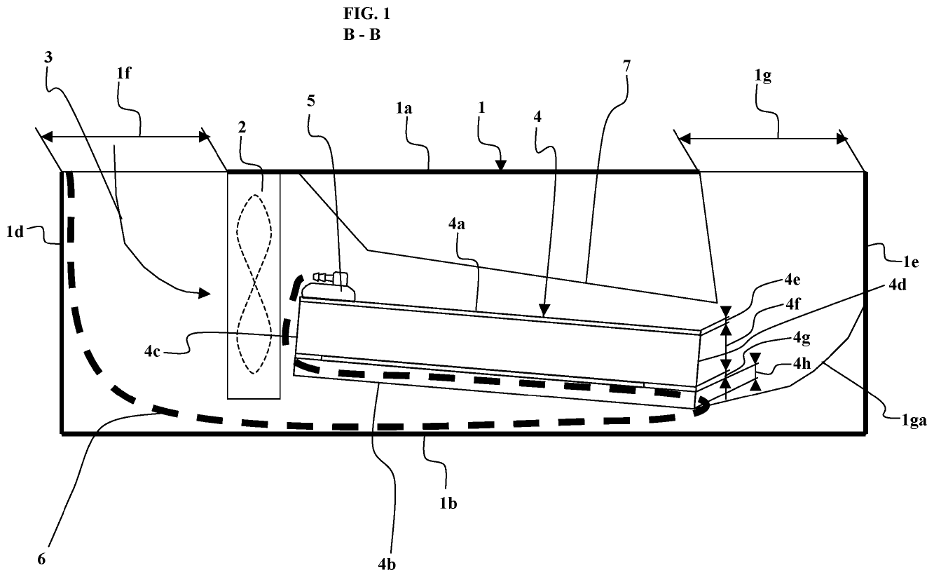

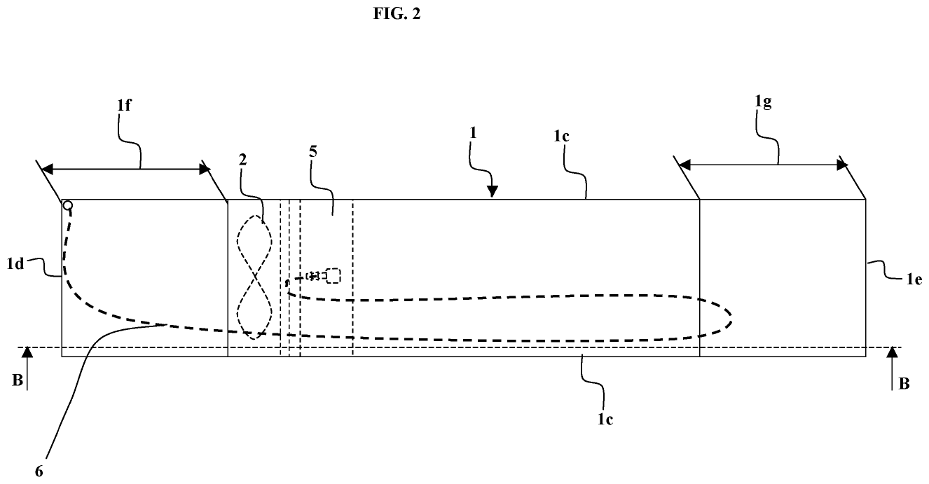

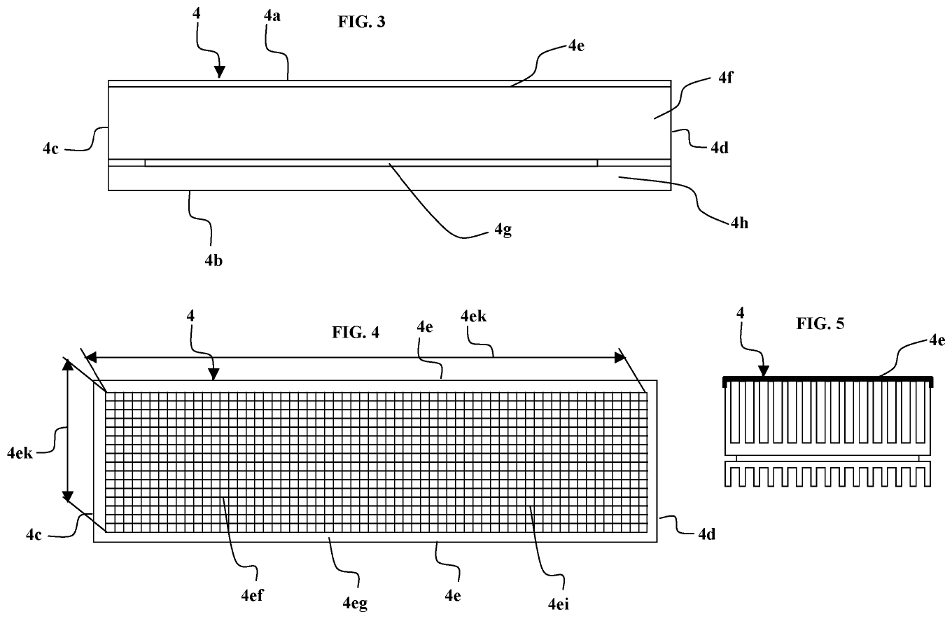

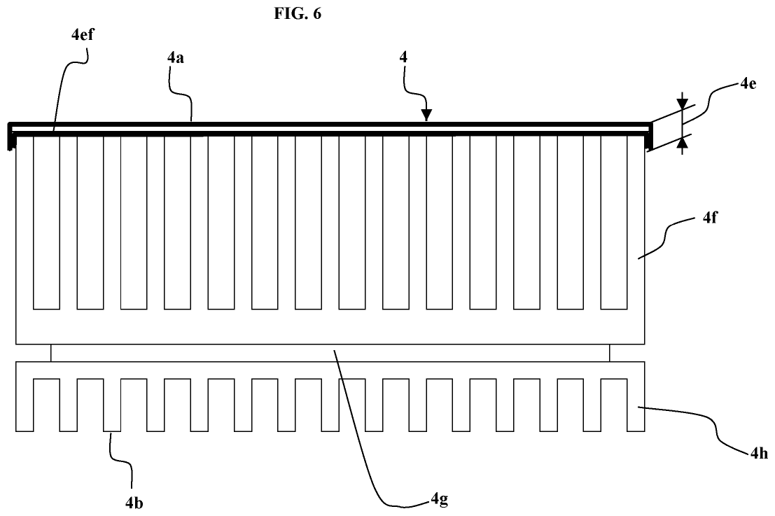

The draining member 6 of hydrogen peroxide is at best a pipe and hose combination so that the starting end of the draining member is of a flexible transparent hose e.g. known from infusion bags of different kind of substances used in hospital technology, and the finishing end of the draining member 6 inside the disinfection device body 1 is of a known metal pipe, such as aluminium pipe, which in accordance with FIGS. 1 and 2, among others, is bent in between the ribs of the warming member 4f and/or the cooler 4h, whereby hydrogen peroxide is pre-warmed as it runs inside the metal pipe towards the draining device 5.

An air guide 7 which directs the airflow from the blower 2 to the fibre-glass braiding 4ef whereby the hydrogen peroxide liquid draining/flowing onto the fibreglass braiding 4ef from the draining device 5 spreads evenly on the fibre-glass braiding 4ef, due to which hydrogen peroxide is gasified efficiently. The air guide 7 also shrinks the air space on top of the fibreglass braiding 4ef whereby the airflow rate on top of the fibreglass braiding 4ef accelerates, a swirling air flow is created, which further speeds up the gasifying of the hydrogen peroxide.

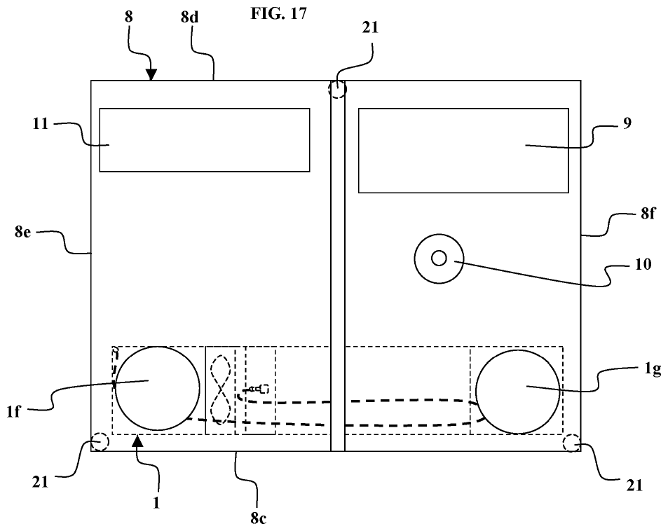

FIG. 17 shows a disinfection device according to the invention the disinfection device body 1 of which is set in a usage space 8, which is a transport/usage briefcase (at best to a device box made of plastic equipped with one or more openable covers)

The usage space 8 has a top side 8a, lower side 8b, front side 8c, rear side 8d, left side 8e, and right side 8f.

The front side 8c, rear side 8d, left side 8e and right side 8f of the usage space 8 are designated only for enabling the description of the invention, they could be referred to by other names too, the locations of the parts of the disinfection device may others than those shown in the Figures.

A partition wall 8g, to which has shown in FIGS. 18 and 19 openable covers 8h, hinged by hinges 8ga which covers 8h are lockable by one or more known latches to a closed and open position, in the open position the covers 8h may be interlocked.

A control apparatus 9 comprises all the control apparatus needed by the disinfection device, and in addition the required connectors to connect electricity, for example, the control apparatus 9 is assembled of known electricity, radio, mobile phone, measurement, control, and communication technology.

A horizontal plane indicator 10 in FIG. 17 is known from bubble levels (an ox-eye bubble level (in the figure in FIG. 17, for example) has a bubble under a convex glass cover which indicates an inclination no matter which compass direction it takes). It may be used for verifying and adjusting the horizontal position of levels, such as tables, with a single glance.) a transparent part, from which by means of an air bubble the position of the usage space 8 may be detected, whereby it is easy to adjust the usage space 8 by known adjustment legs (threaded adjustment paws) on the lower side 8b (by three adjustment legs 21 shown in FIGS. 17 and 18) to a horizontal plane at best there are only three adjustment paws whereby the usage space 8 does not rock. The horizontal position is important for hydrogen peroxide to drain as planned on the evaporation member 4e and to turn into disinfecting hydrogen peroxide gas.

The horizontal plane indicator 10 may also be an electrical horizontal plane indicator 10 manufactured by a prior art technology, an electrical inclination measurement is used among other in battery powered balancing scooters also referred to with name of e-Driftit E-Driftit is a battery-powered vehicle equipped with two wheels, on which vehicle a person stands, and by tilting a person gets the vehicle to move, as well to steer and to stop the vehicle.

In an accessory space 11 among others a hydrogen peroxide bottle/container may be placed, from which hydrogen peroxide is pumped by one or more known electric liquid pumps by means of one or more draining pipes 6 to one or more draining devices 5. As known, the pumping power of a liquid pump is continuously adjustable, whereby the vaporization of hydrogen peroxide may be adjusted to match the environmental conditions and efficiency requirements.

In FIG. 17, the disinfection device body 1 with all the associated parts is placed close to the front side 8c, but its location need not be this.

The suction channel/suction pipe 12 along which suction air 3 may access the disinfection device. The blowing channel/blowing pipe 12 is at best of flexible and continuous so-called wrinkled pipe the length of which may be continued by pulling on and shortened by pressing on the pipe is known among other things from mobile air-conditioning devices.

The blowing channel/blowing pipe 13 along which hydrogen peroxide gas 14 may access the target to be cleaned. The blowing channel/blowing pipe 13 is at best of flexible and continuous so-called wrinkled pipe the length of which may be continued by pulling on and shortened by pressing on the pipe is known among other things from mobile air-conditioning devices.

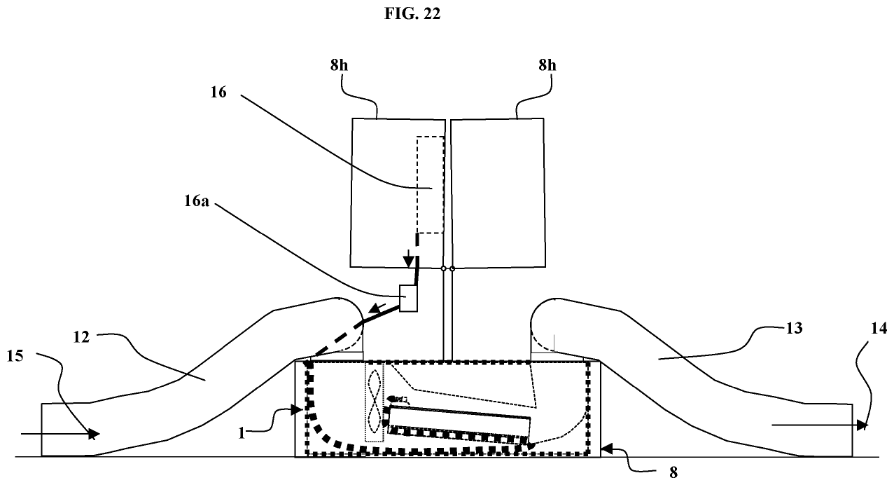

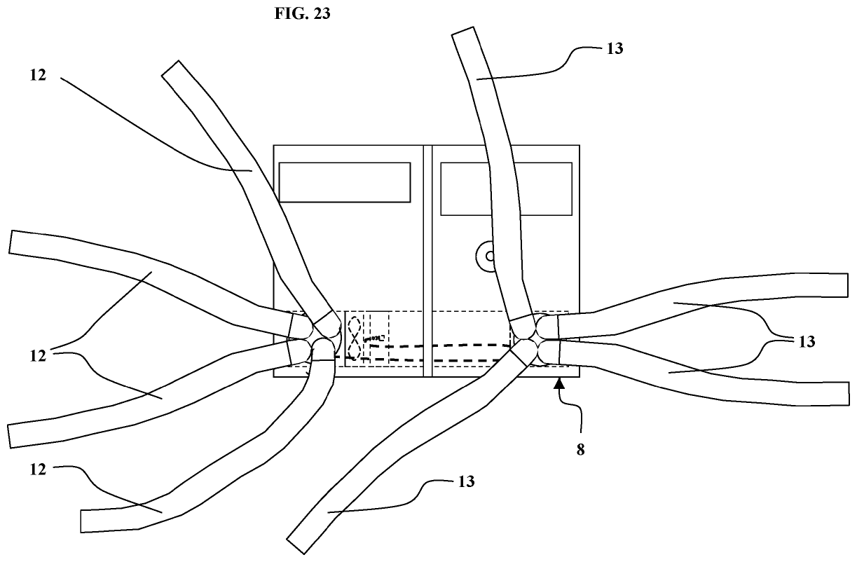

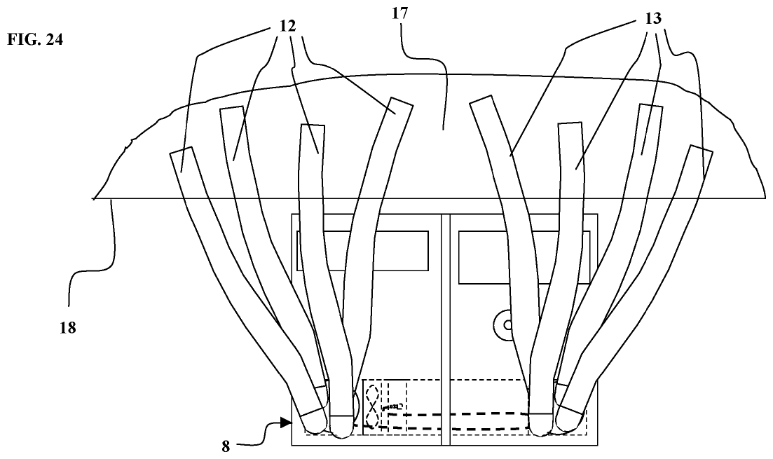

A hydrogen peroxide tank 16, which in FIG. 22 is a hydrogen peroxide infusion bottle or bag, from which liquid hydrogen peroxide drains along the draining pipe 6 to the draining device 5 In the draining pipe 6 in FIG. 22 there is a liquid draining adjusting device 16a known from infusion bags of hospitals.

FIG. 24 shows the disinfection device outside a space 17 to be cleaned, the circulation of air to be cleaned and hydrogen peroxide gas 14 takes place controllably by means of a plurality of pipes. Because the disinfection device is outside the space 17 to be cleaned, the device may be safely serviced, adjusted, and used for the duration of the cleaning. In FIG. 24 the suction channels/suction pipes 12 and the blowing channels/blowing pipes 13 are led through an airtight partitioning wall 18 to the space 17 to be cleaned, the pipes may be sealed with known methods, for example with a suitable seal or tape or the like. The partition wall 18 may be of known tarpaulin, placed in door openings or window openings.

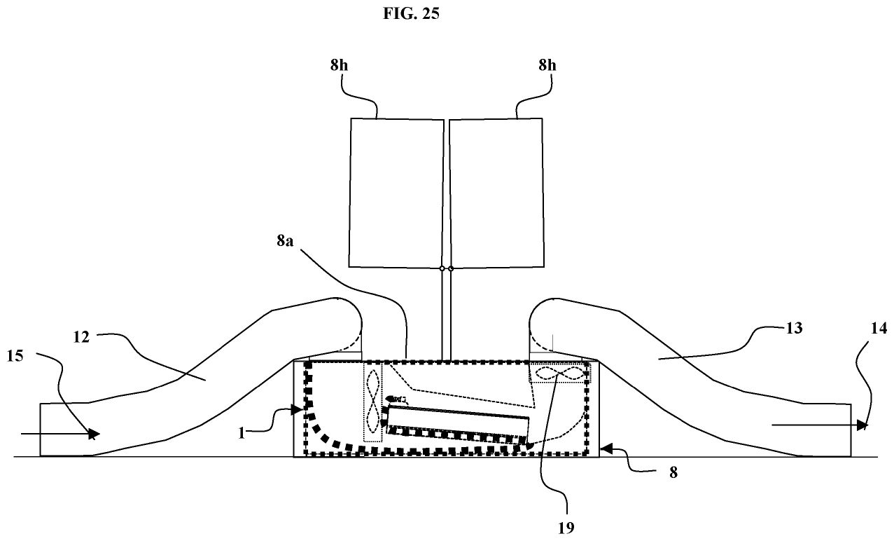

FIG. 25 shows that at the top side la of the disinfection device body 1 at the blowing end le there is one or more pressure blowers 19 in the blowing opening/blowing passage 1g, which boosts the flow of hydrogen peroxide gas 14 in one or more blowing channels/blowing pipes 13.

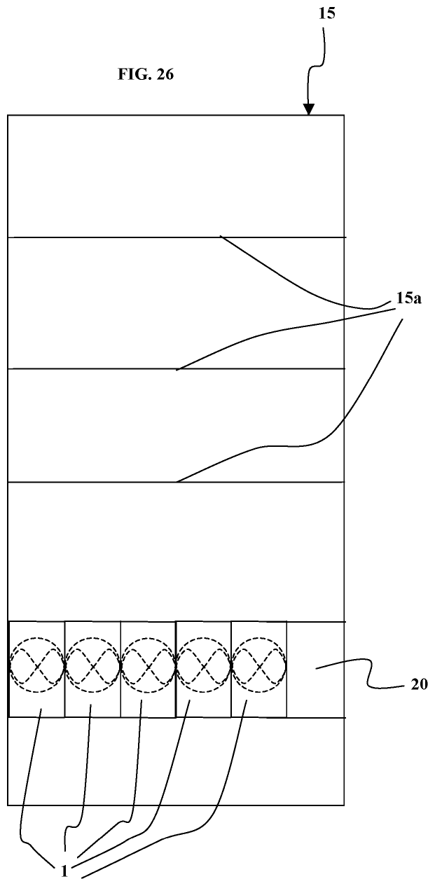

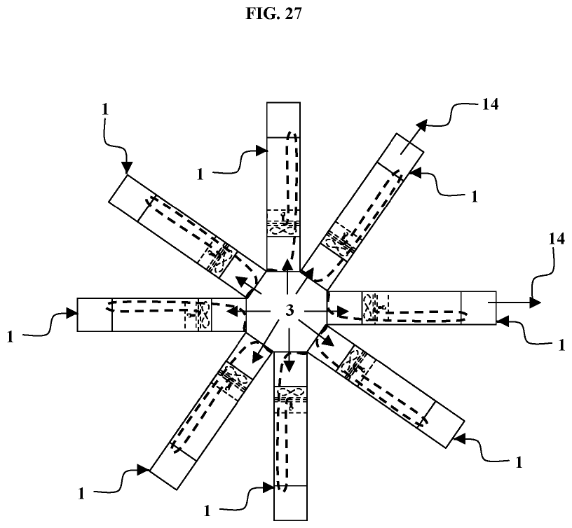

FIG. 26 shows an inventive disinfection cabinet 15 with the front part open or the front part may have a transparent door such as a glass door. The disinfection cabinet 15 has one or more standardised rack spaces 20 for the disinfection device body 1 where the disinfection device body 1 may be put. It is possible to add the desired number of disinfection devices in the disinfection cabinet 15, which may be referred to as VHP units, catalytic converters, air dryers, heaters, etc. may be added to the disinfection cabinet 15 or similar device as standard-sized modules that have standard connectors for electrical connections as well as air inlet and outlet. The modules are “racks” in the same way as an old DIN-sized car radio—each car has an installation place of the same size, which takes in any radio. The production output of the disinfection cabinet 15 is easy to change as needed, because it is simple to add or remove disinfection devices due to the standard rack spaces 20. That is, output is available as a function of the number of rack spaces 20, because a disinfection cabinet has ready-made rack spaces 20 for the disinfection device bodies 1. FIG. 26 shows shelves 15a for the objects or substances/materials to be cleaned, the shelves 15a are preferably grid shelves or grille shelves whereby hydrogen peroxide gas may access any spot inside the disinfection cabinet 15.

In FIG. 27, the disinfection device bodies 1 are placed in a star shape, as seen from the top, whereby hydrogen peroxide gas 14 may spread well to the space to be cleaned, and correspondingly suction air 3 is centrally sucked into the disinfection devices whereby the desired suction air 3 may be guided from the desired place. Suction air 3 may be centrally blown to all the disinfection device bodies 1 by one blower, or alternatively a plurality of blowers may be used and blow suction air 3 along one or more channels/pipers to a hub of disinfection devices bodies 1, shown in FIG. 27. When channels/pipes are used, it is possible to determine precisely from which place suction air 3 is transferred on the disinfection device bodies 1.

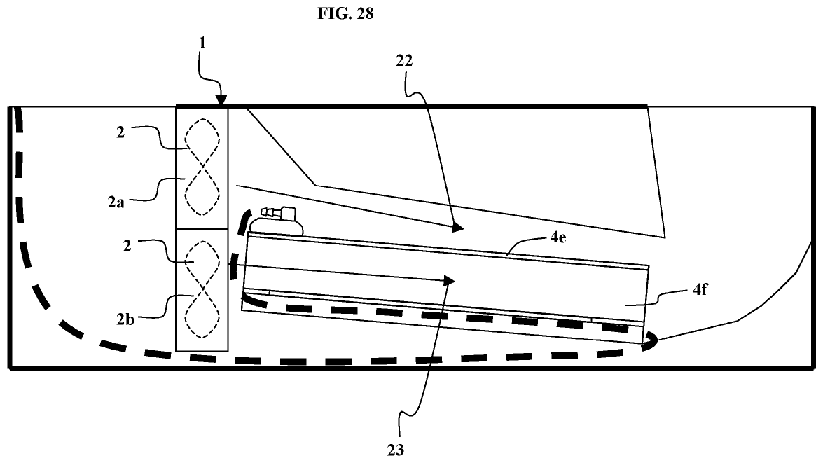

FIG. 28 shows two successive blowers 2. The upper blower 2, called a cold air blower 2a, blows cold air in the direction of the cold air arrow 22 over the evaporation member 4e. The lower blower 2, called a hot air blower 2b, blows air 23 warmed/heated by the warming member 4f, under the evaporation member 4e at a lower rate than the cold air blower 2a above. The blower 2 is the best known axial blower or a similar device moving air, that is, gas. Most preferably filtered air, the filter/cleaner known from the construction industry is not shown in the figures. The gas may be air, nitrogen, argon, or a mixture of the above. The pump 2 is a known pump with which hydrogen superoxide is transferred in liquid form. The pump 2 may be an adjustable-displacement pump or provided with a flow control valve, a separate flow control valve, or a flow control valve internal to the pump.

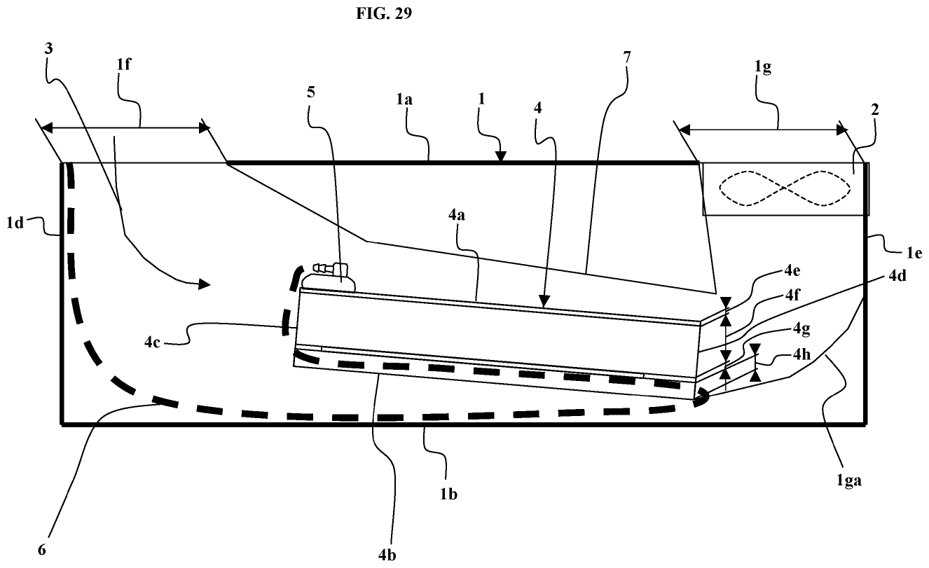

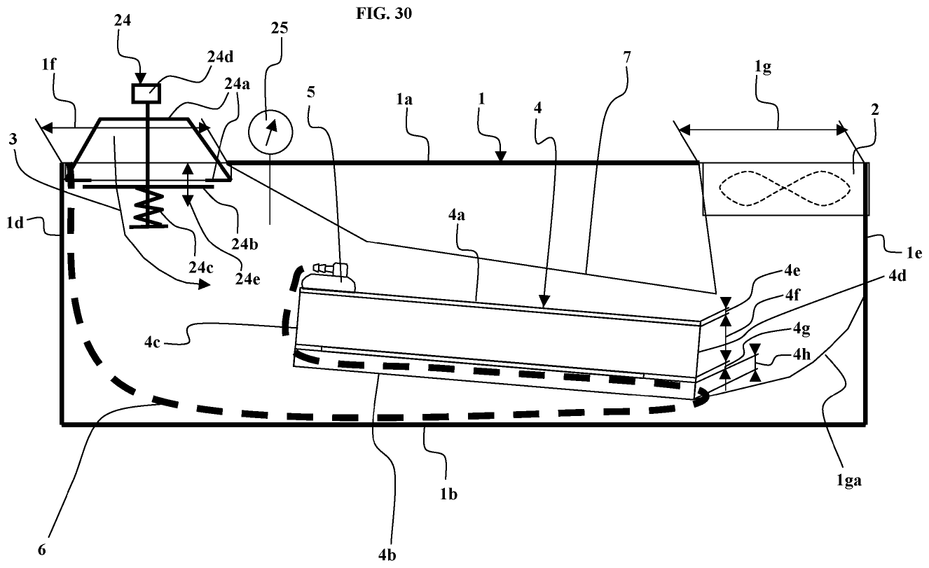

In FIG. 30, an inward relief valve 24 is a spring-loaded disk valve, which opens when the desired vacuum is reached on the spring side of the valve The inward relief valve 24 has a valve body 24a, a closing disk 24b, an adjusting spring 24c (in FIG. 29 the spring is a compression spring), adjustable screw 24d, and an opening/closing direction arrow 24e In FIG. 30, the vacuum gauge is a known vacuum gauge A vacuum lowers the temperature of vaporization of hydrogen peroxide, whereby the vaporization intensifies The shape of the vacuum valve may be other than that shown in FIG. 30, the most important thing is restricting the incoming air to the disinfection device body 1, that is, air pressure is adjusted in the manner the air pressure of apartments is adjusted.

The density/gas content of hydrogen peroxide is 600-800 ppm/m3 (particles/million) in a cubic metre. The disinfection device comprises one or more tanks 16 and one or more pumps 2 to store and transfer hydrogen peroxide (H₂O₂). The pump 2 is an adjustable-displacement pump or in connection with the pump 2 or following it is placed one or more flow control valves by means of which the flow amount of hydrogen peroxide is adjusted for one or more draining devices 5.

The figures show disinfection method to be used for cleaning.

In accordance with the invented method, hydrogen peroxide (H₂O₂) is drained/transferred from one or more tanks 16 by a liquid pressure created by one or more pumps or gravity, through one or more draining pipes 6 to one or more draining devices 5, which draining device 5 drains hydrogen peroxide onto one or more evaporation top surfaces 4ea of an evaporation member 4e of a warming/heating device 4, the evaporation top surface 4ea is at an evaporation angle 4ej of 1 to 30 degrees so that the end on the draining device 5 side is higher, whereby hydrogen peroxide spreads by gravity on the evaporation member 4e where the hydrogen peroxide (H₂O₂) turns into hydrogen peroxide gas due to having been heated by the warming/heating device 4, the disinfection is performed by the hydrogen peroxide gas, a use space 8 partition wall 8b to which hinged covers 8h are attached openable by hinges 8ga, which covers may be locked by one or more known latches to a closed and open position, in the open position the covers 8h may be interlocked.

According to an invented method, at the end on the draining device 5 side there is one or more blowers 2 to blow air in the direction of the evaporation top surface 4ea of the evaporation member 4e of the warming/heating device 4. Airflow is faster on the top side of the evaporation top surface 4ea of the evaporation member 4e of the warming/heating device 4 than on the lower side of the evaporation top surface 4ea.

According to an invented method, the airflow area is reduced by one or more air guides 7 on the top side of the evaporation top surface 4ea of the evaporation member 4e of the warming/heating device 4 towards the gas discharge end 4d whereby the airflow rate is increased towards the gas discharge end 4d.

FIG. 30 shows that with one or more inward relief valves 24 air is restricted from getting inside the disinfection device body 1, whereby as the blower 2 is sucking air from inside the disinfection device body 1, a vacuum is generated inside the disinfection device body 1 A vacuum lowers the temperature of vaporization of hydrogen peroxide, whereby the vaporization intensifies The shape of the vacuum valve may be other than that shown in FIG. 30, the most important thing is restricting the incoming air to the disinfection device body 1, that is, air pressure is adjusted in the manner the air pressure of apartments is adjusted.

In FIG. 30, the inward relief valve 24 is adjustable whereby the desired vacuum may be adjusted inside the disinfection device body 1, the vacuum being measurable by one or more vacuum measurements 25.

The temperature of the airflow on the top side of the evaporation top surface 4ea of the evaporation member 4e of the warming/heating device 4 is lower than on the lower side of the evaporation top surface 4ea.

According to an invented method, the draining device 5 drains hydrogen peroxide to one or more onto one or more evaporation top surfaces 4ea of the evaporation member 4e of the warming/heating device 4, by means of one or more horizontal plane indicators 10 the evaporation top surface 4ea may be adjusted to the correct position in every direction, whereby hydrogen peroxide drains evenly and turns on the evaporation top surface 4ea into hydrogen peroxide gas by means of which hydrogen peroxide gas the disinfection is performed.

According to an invented method, the devices of the disinfection method are located in a disinfection device body 1 which disinfection device body 1 is of a standard size and shape, provided with standard connections, whereby the power of the disinfection method may be selected as desired by placing the desired number of disinfection device bodies 1 in one disinfection device.

The Figures show a disinfection device to be used for cleaning.

In accordance with an invented device, hydrogen peroxide H₂O₂ may be drained/transferred from one or more tanks 16 by a liquid pressure created by one or more pumps or gravity, through one or more draining members 6 to the disinfection device body 1 which has one or more draining devices 5, by means of which draining device 5 hydrogen peroxide may be drained to one or more onto one or more evaporation top surfaces 4ea of an evaporation member 4e of a warming/heating device 4, which evaporation top surface 4ea is at an evaporation angle 4ea of 1 to 30 degrees so that the end on the draining device 5 side is higher, whereby hydrogen peroxide spreads by gravity on the evaporation member 4e where the hydrogen peroxide H₂O₂ turns into hydrogen peroxide gas due to warming by the warming/heating device 4.

According to an invented device, the evaporation member 4e is the top-most part of the warming/heating device 4, on the evaporation top surface 4ea of which to the draining end 4c hydrogen peroxide is drained from one or more draining devices 5, the evaporation member 4e consists of one or more fibreglass braidings, there is one or more nets 4ei on the evaporation member 4e.

According to an invented device, the net 4ei is a net preferably made of metal wire, with a mesh size of 0.3 to 5 mm and wire thickness 0.3 to 1.0 mm. The material of the mesh 4e may be another known heat-resistant material, such as plastic.

According to an invented device by one or more inward relief valves 24 air is restricted from getting inside the disinfection device body 1, whereby as the blower 2 is sucking air from inside the disinfection device body 1, a vacuum is generated inside the disinfection device body 1.

According to an invented device the inward relief valve 24 is adjustable, whereby the desired vacuum may be adjusted inside the disinfection device body 1, which vacuum is measurable by one or more vacuum measurements 25.

AN EXAMPLE OF USING THE INVENTION

The cleaning of surfaces to be disinfected is carried out by one or more invented disinfection devices. Before disinfection is started, the targets to be disinfected must be mechanically cleaned, by a prior art technology, as best as possible, for example foodstuff or other porous targets may be difficult to clean mechanically.

The disinfection device/devices are placed in a closed space, such as a container or room. The room must be possible to be well ventilated, to which a blower and filter unit, known from ventilation of buildings, are connected. In the room, a plurality of air condition management devices are placed to stabilise the humidity level, which are devices known from the management of air condition of premises of buildings, including laboratories where air temperature, humidity, and purity are strictly managed and controlled. The disinfection device/devices are placed in the room. The disinfection device/devices are activated by remote control, using a prior art technology. The disinfection process is automatic and, depending on the room size, takes from a few hours to a day or days, the duration of the process depends on the target being cleaned. After the disinfection, the room is ventilated through known filters to outside air. The cleaned devices/targets are ready for transfer for further measures. The quality assurance documentation may be had from VTT Technical Research Centre of Finland Ltd, for example.

The invented disinfection device may be manufactured by known methods from known materials, most advantageously from metals.

It is apparent to a person skilled in the art that the above exemplary embodiments are rather simple in structure and operation for the purposes of illustration of the description. By following the model shown in this patent application, it is possible to construct different structural solutions that utilise the inventive idea disclosed in this patent application. The invention is not restricted to the alternatives disclosed in the above, but many variations are possible within the scope of the inventive idea defined by the attached claims.

1. A disinfection method for use for cleaning, wherein hydrogen peroxide (H₂O₂) is drained/transferred from one or more tanks by a liquid pressure created by one or more pumps or gravity, through one or more draining members to one or more draining devices, which draining device drains hydrogen peroxide to one or more onto one or more evaporation top surfaces of an evaporation member of a warming/heating device, the evaporation top surface is at an evaporation angle of 1 to 30 degrees in relation to the horizontal plane so that the end on the draining device side, i.e. a draining end, is higher than an opposite end of the draining device of the warming/heating device, i.e. a gas discharge end, whereby hydrogen peroxide spreads by gravity on the evaporation member where the hydrogen peroxide (H₂O₂) turns into hydrogen peroxide gas, by means of which hydrogen peroxide gas the disinfection is performed, the evaporation member consists of one or more fibreglass braidings, the airflow area is reduced by one or more air guides on the top side of the evaporation top surface of the evaporation member of the warming/heating device towards the gas discharge end whereby the airflow rate is increased towards the gas discharge end.

2. The disinfection method of claim 1, wherein the end on the draining device side has one or more blowers to blow air in the direction of the evaporation top surface of the evaporation member of the warming/heating device.

3. The disinfection method of claim 1, wherein suction air is sucked by one or more blowers, whereby a vacuum is formed on the evaporation top surface, due to which vacuum hydrogen peroxide turns into hydrogen peroxide gas at a low temperature.

4. The disinfection method of claim 3, wherein with one or more inward relief valves air is restricted from getting inside the disinfection device body, whereby as the blower is sucking air from inside the disinfection device body, a vacuum is generated inside the disinfection device body.

5. The disinfection method of claim 1, wherein airflow is faster on the top side of the evaporation top surface of the evaporation member of the warming/heating device than on the lower side of the evaporation top surface, the disinfection method comprises two successive blowers, the upper blower, called a cold air blower, blows cold air in the direction of the cold air arrow over the evaporation member, the lower blower, called a hot air blower blows air warmed/heated by the warming member, under the evaporation member at a lower rate than the cold air blower above.

6. The disinfection method of claim 1, wherein a use space partition wall to which hinged covers are attached openable by hinges, which covers may be locked by one or more known latches to a closed and open position, in the open position the covers may be interlocked.

7. The disinfection method of claim 1, wherein the temperature of the airflow on the top side of the evaporation top surface of the evaporation member of the warming/heating device is lower than on the lower side of the evaporation top surface.

8. The disinfection method of claim 1, wherein the draining device drains hydrogen peroxide to one or more onto one or more evaporation top surfaces of the evaporation member of the warming/heating device, by means of a horizontal plane indicators the evaporation top surface may be adjusted to the correct position in every direction, whereby hydrogen peroxide drains evenly and turns into hydrogen peroxide gas on the evaporation top surface with which hydrogen peroxide gas the disinfection is performed.

9. The disinfection method of claim 1, wherein the devices of the disinfection method are located in a disinfection device body which disinfection device body is of a standard size and shape, provided with standard connections, whereby the power of the disinfection method may be selected as desired by placing the desired number of disinfection device bodies in one disinfection device.

10. A disinfection device for use for cleaning, wherein hydrogen peroxide (H₂O₂) may be drained/transferred from one or more tanks by a liquid pressure created by one or more pumps or gravity, through one or more draining members to a draining device body which has one or more draining devices, by means of which draining device hydrogen peroxide may be drained to one or more onto one or more evaporation top surfaces of an evaporation member of a warming/heating device, the evaporation top surface is at an evaporation angle of 1 to 30 degrees in relation to the horizontal plane so that the end on the draining device side, i.e. a draining end, is higher than an opposite end of the draining device of the warming/heating device, i.e. a gas discharge end, whereby hydrogen peroxide spreads by gravity on the evaporation member where the hydrogen peroxide (H₂O₂) turns into hydrogen peroxide gas, by means of which hydrogen peroxide gas the disinfection is performed, the evaporation member consists of one or more fibreglass braidings, the airflow area is reduced by one or more air guides on the top side of the evaporation top surface of the evaporation member of the warming/heating device towards the gas discharge end whereby the airflow rate is increased towards the gas discharge end.

11. The disinfection device of claim 10, wherein the evaporation member is the topmost part of the warming/heating device, on the evaporation top surface of which on the draining end hydrogen peroxide is drained from one or more draining devices, the evaporation member consists of one or more fibreglass braidings, there is one or more nets on the evaporation member.

12. The disinfection device of claim 10, wherein the net is a net made of metal wire, with a mesh size of 0.3 to 5 mm and wire thickness 0.3 to 1.0 mm.

13. The disinfection device of claim 10, wherein one or more inward relief valves are used to restrict air from getting inside the disinfection device body, whereby as the blower is sucking air from inside the disinfection device body, a vacuum is generated inside the disinfection device body.

14. The disinfection device of claim 13, wherein the inward relief valve- is adjustable, whereby the desired vacuum may be adjusted inside the disinfection device body, the vacuum being measurable by one or more vacuum gauges.
